(19) United States
(12) Patent Application Publication
(10) Pub. No.: US 2025/0052653 A1
(43) Pub. Date: Feb. 13, 2025

(54) FOUNDRY SAND INSPECTION METHOD AND FOUNDRY SAND INSPECTION DEVICE

(71) Applicant: SINTOKOGIO, LTD., Nagoya-shi (JP)

(72) Inventor: Yuichi OGURA, Nagoya-shi (JP)

(73) Assignee: SINTOKOGIO, LTD., Nagoya-shi (JP)

(21) Appl. No.: 18/768,534

(22) Filed: Jul. 10, 2024

(30) Foreign Application Priority Data
Aug. 7, 2023 (JP) 2023-128629

(51) Int. Cl. G01N 3/08; G01N 33/24

(52) U.S. Cl. CPC G01N 3/08; G01N 33/24; G01N 2203/0019

(57) ABSTRACT

A foundry sand inspection method according to an aspect includes: charging foundry sand into a test tube; measuring compactability (CB) of the foundry sand while compressing the foundry sand in the test tube to produce a test piece; and compressing the test piece and measuring a compressive strength of the test piece.

### CROSS-REFERENCE TO RELATED APPLICATIONS

This application is based on and claims the benefit of priority from Japanese Patent Application No. 2023-128629 filed on Aug. 7, 2023, the entire contents of which are incorporated herein by reference.

### TECHNICAL FIELD

The present disclosure relates to a foundry sand inspection method and a foundry sand inspection device.

### BACKGROUND

Sand molds obtained by molding foundry sand have been widely used as molds for casting. For example, a green sand mold, which is a type of sand mold, is produced by using foundry sand to which a binder and an additive to silica sand have been added. Typically, green sand molds used for casting are crushed to return to foundry sand, and then, a binder and an additive are added to the recovered foundry sand to enable the sand to be reused as recycled sand. Since the quality of the recycled foundry sand affects the accuracy of casting, the quality of the foundry sand is controlled in foundry mills. Specifically, parameters indicating characteristics of the foundry sand, such as compactability (CB), moisture content, temperature, air permeability, and compressive strength, are measured before molding of the sand mold so as to confirm whether these parameters are within control values.

Examples of known techniques for inspecting foundry sand include a device described in Japanese Unexamined Patent Publication No. H9-318511. The device described in Japanese Unexamined Patent Publication No. H9-318511 includes a test tube, a pressing rod having a pressurizing plate that moves up and down in the test tube, and a cylinder having a piston rod coupled to the pressing rod. After the pressurizing plate is positioned based on pre-measured CB of foundry sand, the foundry sand is supplied into the test tube. The foundry sand is compressed in the test tube to produce a test piece having a predetermined height (50 mm). The test piece is compressed at a predetermined pressure, and a compressive strength of the test piece is measured from a load at the breakage of the test piece.

### SUMMARY

### Technical Problem

As described above, the device described in Japanese Unexamined Patent Publication No. H9-318511 pre-measures the CB of foundry sand in order to produce a test piece having a predetermined height, and thereafter measures the compressive strength. That is, with this device, a sample of the foundry sand is charged in two sessions, and the CB and the compressive strength of the foundry sand are individually measured from each sample, having a problem of a prolonged inspection time of the foundry sand.

In view of this, an object of the present disclosure is to shorten an inspection time of foundry sand.

### Solution to Problem

A foundry sand inspection method according to an aspect includes: charging foundry sand into a test tube; measuring compactability (CB) of the foundry sand while compressing the foundry sand in the test tube to produce a test piece; and compressing the test piece and measuring a compressive strength of the test piece.

### Advantageous Effects of Invention

According to the present disclosure, it is possible to shorten an inspection time of foundry sand.

### BRIEF DESCRIPTION OF THE DRAWINGS

FIG. 1 is a diagram schematically illustrating a configuration of an inspection device according to an embodiment;

FIG. 2 is a block diagram illustrating a functional configuration of a control device;

FIG. 3 is a flowchart illustrating an inspection method according to an embodiment;

FIG. 4A, FIG. 4B and FIG. 4C are a diagram illustrating an operation of the inspection device;

FIG. 5A, FIG. 5B and FIG. 5C are a diagram illustrating an operation of the inspection device;

FIG. 6A and FIG. 6B are a diagram illustrating an operation of the inspection device;

FIG. 7 is a graph illustrating a relationship between a height of a test piece and compressive strength; and

FIG. 8 is a graph illustrating a relationship between compressive strength before correction and compressive strength after correction.

### DETAILED DESCRIPTION

Hereinafter, embodiments of the present disclosure will be described in detail with reference to the drawings. In the description of the drawings, the same elements are denoted by the same reference signs, and redundant description is omitted. The drawings are sometimes partially simplified or exaggerated for easy understanding, and dimensional ratios, angles, and the like are not limited to those described in the drawings. In the present specification, the words “upper”, “lower”, “left”, and “right” are based on the illustrated state and are used for convenience.

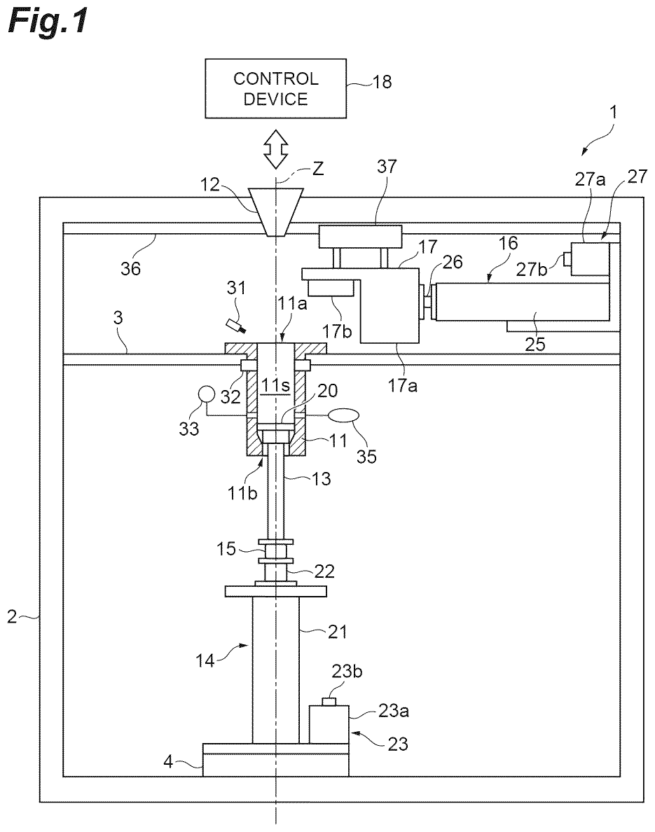

FIG. 1 is a diagram schematically illustrating a configuration of an inspection device 1 according to an embodiment. The inspection device 1 inspects characteristics of foundry sand used for the molding of molds. Foundry sand is a raw material for sand molds, and is typically green sand. Green sand is produced by kneading a mixture of silica sand, a binder, and an additive, for example. An example of the binder to be used for green molds is bentonite. Examples of the additive to be used include water, coal powder, and starch.

The accuracy of the casting is affected by characteristics of the foundry sand, and thus, the characteristics of the foundry sand are inspected before molding of the mold. Examples of parameters representing characteristics of the foundry sand include compactability (CB), moisture content, temperature, air permeability, and compressive strength. The parameters representing the characteristics of the foundry sand are measured and stored by the inspection device 1. When a defect of the casting occurs, the stored parameter is read and used for an action for suppressing the defect of the casting.

CB is the shrinkage ratio [%] when the foundry sand is compressed with a constant strength. The moisture content represents free moisture content in the foundry sand. The compressive strength [N/cm²] is a value obtained by dividing a load when a standard test piece is compressed to be broken by a cross-sectional area of the standard test piece. The standard test piece is produced by a method prescribed in JIS Z 2601 “Methods for determining foundry molding sand properties”. Specifically, JIS Z 2601 specifies a method in which foundry sand in a test tube is compacted at a predetermined pressure to produce a standard test piece having a height of 50 mm+1 mm, and the compressive strength is measured using the standard test piece.

As illustrated in FIG. 1, the inspection device 1 includes a test tube 11, a chute 12, a pressing rod 13, a first cylinder 14, a load sensor 15, a second cylinder 16, a scraper 17, and a control device 18. The test tube 11 is a cylindrical tubular body made of steel, for example, and defines an internal space 11s for accommodating foundry sand. The test tube 11 is supported by a frame 2 via a support member 3. The test tube 11 has an upper opening 11a and a lower opening 11b, and extends along an axis Z between the upper opening 11a and the lower opening 11b. Hereinafter, the direction along the axis Z may be referred to as an “axial direction”. Typically, the axial direction matches the vertical direction. The inner diameter of the test tube 11 is constant in the axial direction.

The chute 12 is provided above the test tube 11. The chute 12 guides foundry sand stored in a tank (not illustrated) to be charged into the test tube 11. The chute 12 may have an openable and closable discharge port so as to be able to switch supply/stop of foundry sand to the test tube 11 by opening and closing the discharge port. The opening and closing of the discharge port is controlled by the control device 18.

The pressing rod 13 extends along the axis Z, and has its upper portion inserted into the test tube 11. The pressing rod 13 is provided, at its upper end, with a pressurizing plate 20. The pressurizing plate 20 is a disk-shaped plate, for example, and is disposed in the internal space 11s of the test tube 11. The lower end of the pressing rod 13 is connected to a piston rod 22 of a first cylinder 14 described below. The pressing rod 13 moves in the axial direction together with the up-down movement operation of the piston rod 22.

The first cylinder 14 is provided below the test tube 11. The first cylinder 14 includes a cylinder body 21, a piston rod 22, and a motor 23. The cylinder body 21 is disposed on a mount 4 disposed at the bottom of the inspection device 1, and supports the piston rod 22 so as to be movable forward and backward. The piston rod 22 extends upward from the cylinder body 21 along the axis Z, and is provided so as to be movable up and down with respect to the cylinder body 21. An upper end of the piston rod 22 is connected to a lower end of the pressing rod 13. The pressurizing plate 20 of the pressing rod 13 moves in the axial direction (vertical direction) in the test tube 11 together with the up-down movement of the piston rod 22.

The motor 23 is a servomotor that allows up-down movement of the piston rod 22, for example. The motor 23 includes a motor body 23a and an encoder 23b. The motor body 23a drives an output shaft by power supplied from an external power supply. The driving force of the motor body 23a is transmitted to the piston rod 22 via the cylinder body 21 so as to allow the piston rod 22 to move up and down along the axis Z. The encoder 23b detects a rotation angle of the output shaft of the motor body 23a. The rotation angle of the output shaft corresponds to a moving distance of the pressing rod 13 in the axial direction. The encoder 23b specifies the position of the pressurizing plate 20 in the axial direction based on the rotation angle of the output shaft.

The load sensor 15 is disposed between the pressing rod 13 and the piston rod 22. The load sensor 15 is implemented by a load cell, for example, and measures a load applied from the piston rod 22 to the pressing rod 13. The load sensor 15 outputs information indicating the measured load to the control device 18.

The second cylinder 16 is provided on the side of the test tube 11. The second cylinder 16 includes a cylinder body 25, a piston rod 26, and a motor 27. The cylinder body 25 is supported by the frame 2, and supports the piston rod 26 so as to be movable forward and backward in a direction (horizontal direction) perpendicular to the axis Z. The piston rod 26 extends in the horizontal direction from the cylinder body 25 and is provided to be movable forward and backward with respect to the cylinder body 25. The tip of the piston rod 26 is coupled to the scraper 17. The scraper 17 moves in the horizontal direction together with forward and backward movements of the piston rod 26.

The motor 27 is a servomotor that drives the piston rod 26, for example. The motor 27 includes a motor body 27a and an encoder 27b. The motor body 27a drives an output shaft by power supplied from an external power supply. The driving force of the motor body 27a is transmitted to the piston rod 26 via the cylinder body 25 so as to move the piston rod 26 in the horizontal direction. The encoder 27b detects a rotation angle of the output shaft of the motor body 27a. The rotation angle of the output shaft corresponds to a moving distance of the scraper 17 in the horizontal direction. The encoder 27b specifies the position of the scraper 17 in the horizontal direction based on the rotation angle of the output shaft.

The scraper 17 is coupled to the piston rod 26 of the second cylinder 16. The scraper 17 includes: a closing surface 17a for closing the upper opening 11a of the test tube 11; and a measuring surface 17b disposed above the closing surface 17a. The closing surface 17a and the measuring surface 17b are planes perpendicular to the axis Z and face downward. The closing surface 17a is disposed at substantially the same height position as the upper opening 11a of the test tube 11 in the axial direction. The distance between the closing surface 17a and the measuring surface 17b in the axial direction is set to be higher than the target height of the test piece.

The scraper 17 moves in the horizontal direction together with forward and backward movements of the piston rod 26. Specifically, the scraper 17 is movable between positions including: a closing position in which the closing surface 17a closes the upper opening 11a of the test tube 11; a retracted position offset with respect to a foundry sand supply path from the chute 12 to the test tube 11; and a measurement position in which the measuring surface 17b is disposed above the upper opening 11a of the test tube 11.

The inspection device 1 may further include a rail 36 and a slider 37. The rail 36 is fixed to the upper portion of the frame 2 and extends parallel to the piston rod 26. The slider 37 is slidably attached to the rail 36. The slider 37 is coupled to the scraper 17 by bolts or the like. When the piston rod 26 moves forward or backward, the slider 37 slides along the rail 36 to guide the scraper 17 in the horizontal direction.

The inspection device 1 also includes various sensors for measuring characteristics of the foundry sand. Specifically, the inspection device 1 includes a temperature sensor 31, a moisture sensor 32, and a pressure sensor 33. The temperature sensor 31 is disposed above the test tube 11 and measures the temperature of the foundry sand. The moisture sensor 32 is provided in the test tube 11, and measures the moisture content of the foundry sand accommodated in the internal space 11s of the test tube 11. The pressure sensor 33 is provided in the test tube 11 and measures the pressure in the internal space 11s of the test tube 11.

The control device 18 is a computer including a processor, a storage unit, an input device, a display device, and the like, and controls the entire operation of the inspection device 1. The storage unit of the control device 18 stores a control program for causing the processor to control various processes executed by the control device 18. The control device 18 is communicably connected to the chute 12, the first cylinder 14, the load sensor 15, the second cylinder 16, the encoders 23b and 27b, the temperature sensor 31, the moisture sensor 32, and the pressure sensor 33.

The control device 18 transmits a control signal to the chute 12, the first cylinder 14, and the second cylinder 16 to control operations of the chute 12, the first cylinder 14, and the second cylinder 16. Furthermore, the control device 18 receives measured values of the load sensor 15, the temperature sensor 31, the moisture sensor 32, and the pressure sensor 33 so as to measure parameters indicating characteristics of the foundry sand.

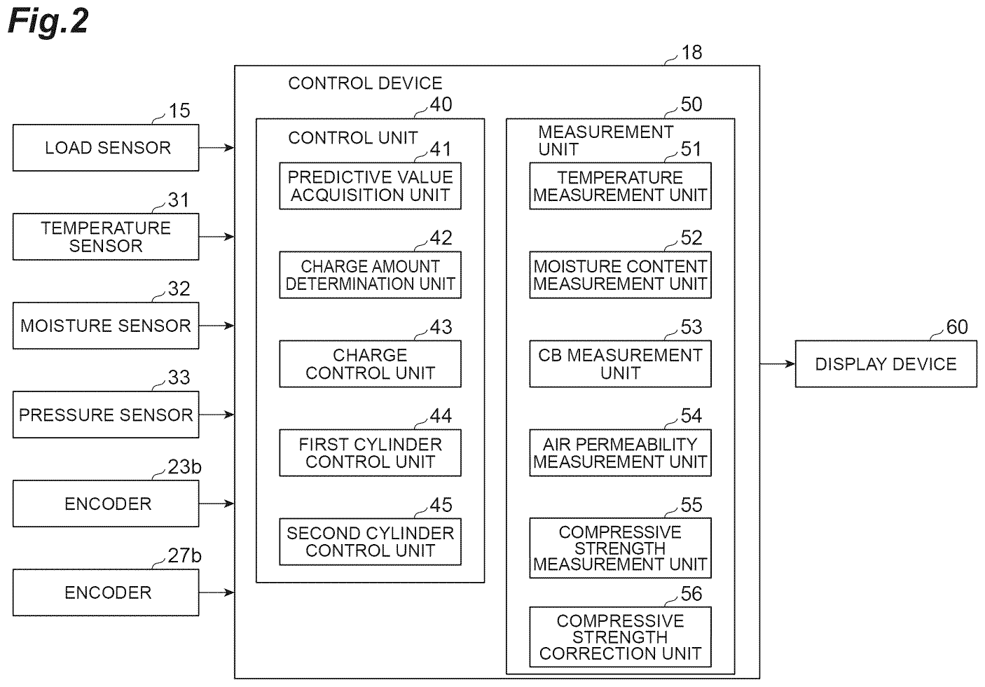

FIG. 2 is a block diagram illustrating a functional configuration of the control device 18. As illustrated in FIG. 2, the control device 18 includes a control unit 40 and a measurement unit 50 as functional configurations. The control unit 40 includes a predictive value acquisition unit 41, a charge amount determination unit 42, a charge control unit 43, a first cylinder control unit 44, and a second cylinder control unit 45.

The predictive value acquisition unit 41 acquires a predictive value of CB of foundry sand. In some cases, a skilled operator can predict CB of foundry sand from the appearance, texture, weather, and the like of the foundry sand. In this case, the predictive value acquisition unit 41 acquires a predictive value of the CB input from the operator via the input device, for example. Alternatively, the predictive value acquisition unit 41 may acquire CB of foundry sand measured in the past, as a predictive value of CB. For example, the CB of the foundry sand measured in the past for each processing line of the foundry sand is stored in the storage unit, and thus, the predictive value acquisition unit 41 may read the CB of the corresponding processing line from the storage unit and acquire the CB as the predictive value of the CB.

The charge amount determination unit 42 determines the amount of foundry sand to be charged into the test tube 11 in order to produce a test piece having a target height (for example, 50 mm). The amount of foundry sand to be charged into the test tube 11 is obtained from the target height of the test piece to be produced and the predictive value of the CB. The amount of foundry sand to be charged into the test tube 11 is represented by the height of the foundry sand before compression. The height of the foundry sand before compression is the height of the foundry sand before compression in the test tube 11 based on the height of the pressurizing plate 20 as a reference. In the following description, the height of the foundry sand before compression may be referred to as a “initial sand height”. For example, when the target height of the test piece is Ht and the predictive value of CB is CBp, an initial sand height H1 of the test tube 11 is calculated as in the following Formula (1).

H1=100×Ht/(100−CBp) (1)

The initial sand height H1 obtained by Formula (1) corresponds to the amount of foundry sand to be charged into the test tube 11 in order to produce a test piece having the target height Ht. As described above, the lower the predictive value CBp of the CB, the lower the initial sand height H1 will be, and the higher the predictive value CBp of the CB, the higher the initial sand height H1 will be. Note that the predictive value acquisition unit 41 may acquire, from an operator, a predictive value of the amount of foundry sand to be charged into the test tube 11 in order to produce a test piece having the target height Ht. When the predictive value of the amount of foundry sand has been input by the operator, the predictive value may be determined as the amount of foundry sand to be charged into the test tube 11 without acquiring the predictive value of CB.

The charge control unit 43 controls the chute 12 to supply foundry sand into the test tube 11. For example, the charge control unit 43 controls the chute 12 so as to allow the amount of foundry sand determined by the charge amount determination unit 42 to be charged into the test tube 11.

The first cylinder control unit 44 controls the motor 23 of the first cylinder 14 to adjust the stroke amount (upward-moving distance or downward-moving distance) of the piston rod 22. The position of the pressurizing plate 20 in the axial direction is adjusted according to the stroke amount of the piston rod 22. The position of the pressurizing plate 20 in the axial direction is specified based on the output of the encoder 23b. That is, the first cylinder control unit 44 controls the position of the pressurizing plate 20 in the vertical direction based on the output of the encoder 23b. The depth of the test tube 11, that is, the initial sand height H1 changes according to the position of the pressurizing plate 20 in the axial direction.

The second cylinder control unit 45 controls the motor 27 of the second cylinder 16 to adjust the stroke amount of the piston rod 26. The position of the scraper 17 in the horizontal direction is adjusted according to the stroke amount of the piston rod 26. The position of the scraper 17 in the horizontal direction is specified based on the output of the encoder 27b. That is, the second cylinder control unit 45 controls the position of the scraper 17 in the horizontal direction based on the output of the encoder 27b.

The measurement unit 50 includes a temperature measurement unit 51, a moisture content measurement unit 52, a CB measurement unit 53, an air permeability measurement unit 54, a compressive strength measurement unit 55, and a compressive strength correction unit 56. The temperature measurement unit 51 measures the temperature of the foundry sand based on the output value of the temperature sensor 31. The moisture content measurement unit 52 measures the moisture content of the foundry sand based on the output value of the moisture sensor 32.

The CB measurement unit 53 measures the CB of the foundry sand based on a difference between the height of the foundry sand in the test tube 11 before compression and the height of the foundry sand in the test tube 11 after compression. The height of the foundry sand before compression is the surface height of the foundry sand in the test tube 11 based on the height of the pressurizing plate 20, that is, the initial sand height. The height of the foundry sand after compression is the height of a test piece produced by compressing the foundry sand. The height of the foundry sand in the test tube 11 is specified based on the stroke amount of the first cylinder 14 determined by the output of the encoder 23b.

The air permeability measurement unit 54 measures air permeability of the test piece. Specifically, the air permeability measurement unit 54 supplies air from an air source 35 into the test tube 11 so as to allow the air to pass through the test piece, and measures the air permeability of the test piece based on the flow rate of the air when the pressure in the test tube 11 becomes constant.

The compressive strength measurement unit 55 measures the compressive strength of the test piece. Specifically, the compressive strength measurement unit 55 acquires, from the load sensor 15, a load when the test piece is compressed to be broken, and measures the compressive strength of the test piece based on the obtained load.

The compressive strength correction unit 56 corrects the compressive strength based on a difference between the target height of the test piece and the measured value of the height of the test piece. For example, when the measured value of the height of the test piece is greater than the target height, the compressive strength correction unit 56 corrects the compressive strength to increase the measured compressive strength, and when the measured value of the height of the test piece is smaller than the target height, the compressive strength correction unit 56 corrects the compressive strength to decrease the measured compressive strength. The measurement unit 50 stores measured information regarding the foundry sand, such as the temperature, moisture content, CB, air permeability, and corrected compressive strength, in the storage unit while displaying the information on a display device 60.

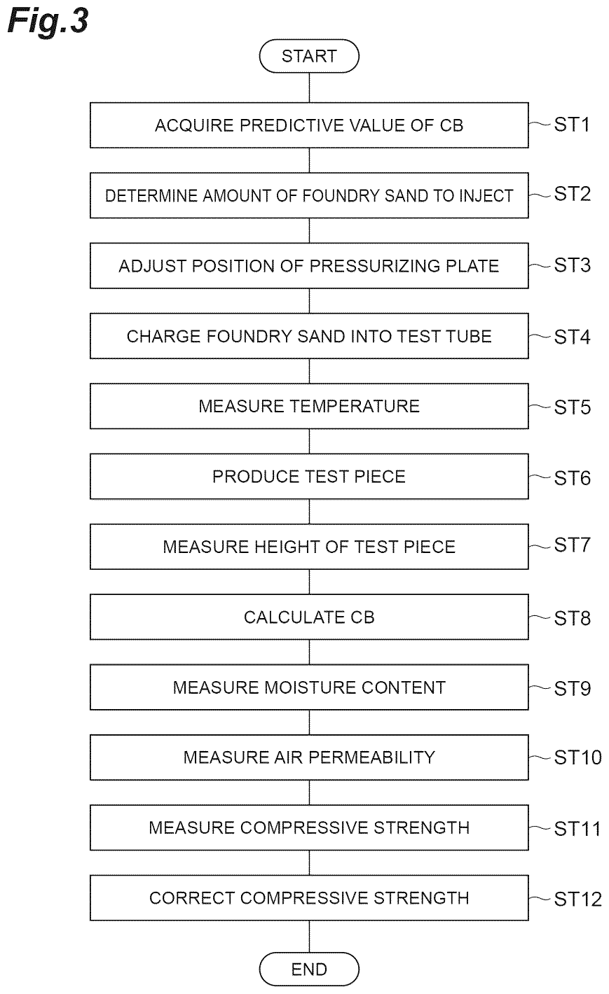

Hereinafter, a foundry sand inspection method using the inspection device 1 will be described. FIG. 3 is a flowchart illustrating a foundry sand inspection method according to an embodiment. This inspection method, measures CB and compressive strength of foundry sand S1 charged into the test tube 11. Each step of FIG. 3 is executed by the control device 18 controlling each component of the inspection device 1.

As illustrated in FIG. 3, the predictive value acquisition unit 41 of the control device 18 first acquires a predictive value of CB of the foundry sand S1 (step ST1). The predictive value of CB is input to the inspection device 1 by an operator, for example. The predictive value acquisition unit 41 may read CB of the foundry sand S1 measured in the past from the storage unit and use the read CB as the predictive value of CB.

Next, the charge amount determination unit 42 determines the amount of the foundry sand S1 to be charged into the test tube 11 in order to produce a test piece S2 having the target height (for example, 50 mm) (step ST2). For example, the charge amount determination unit 42 assigns the target height of the test piece S2 and the predictive value of the CB into the above Formula (1) to obtain the initial sand height H1. As described above, since the diameter of the test tube 11 is constant in the axial direction, the initial sand height H1 corresponds to the amount of foundry sand S1 to be charged into the test tube 11 in order to produce the test piece S2 having a target height.

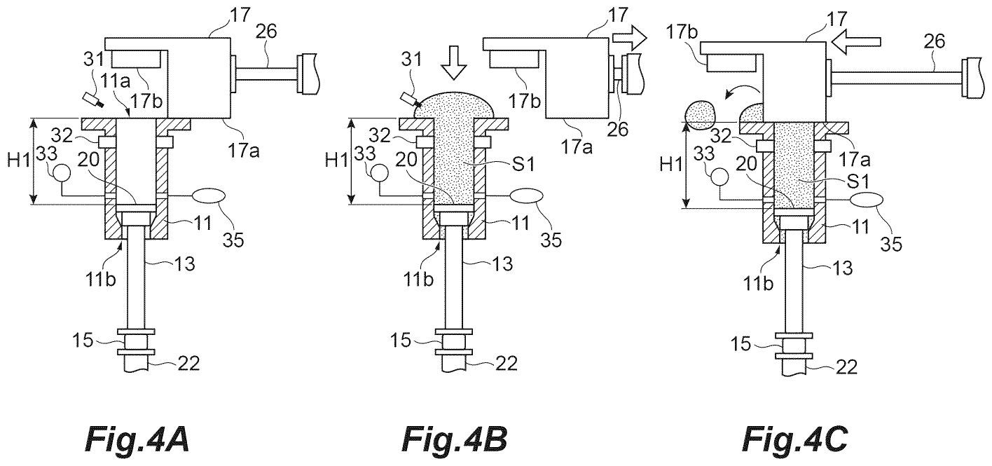

Next, as illustrated in FIG. 4A, the first cylinder control unit 44 controls the stroke amount of the first cylinder 14 to adjust the position of the pressurizing plate 20 so that the distance between the pressurizing plate 20 and the upper opening 11a of the test tube 11 matches the initial sand height H1 obtained in step ST2 (step ST3). Next, as illustrated in FIG. 4B, the second cylinder control unit 45 controls the second cylinder 16 to dispose the scraper 17 at the retracted position. Subsequently, the charge control unit 43 controls the chute 12 to charge the foundry sand S1 into the test tube 11 (step ST4). At this time, the chute 12 charges the foundry sand S1 into the test tube 11 such that the foundry sand S1 overflows from the upper opening 11a of the test tube 11.

Next, the temperature sensor 31 measures the temperature of the foundry sand S1 (step ST5). The temperature measurement unit 51 acquires information indicating the temperature of the foundry sand S1 from the temperature sensor 31 and stores the acquired information.

Next, as illustrated in FIG. 4C, the second cylinder control unit 45 controls the piston rod 26 of the second cylinder 16 to move forward to dispose the scraper 17 at the closing position. The movement of the scraper 17 from the retracted position to the closing position removes a part of the excess foundry sand S1 overflowing from the test tube 11. When the scraper 17 is disposed at the closing position, the upper opening 11a of the test tube 11 is covered and closed with the closing surface 17a of the scraper 17. With this operation, the height of the foundry sand S1 before compression matches the initial sand height H1.

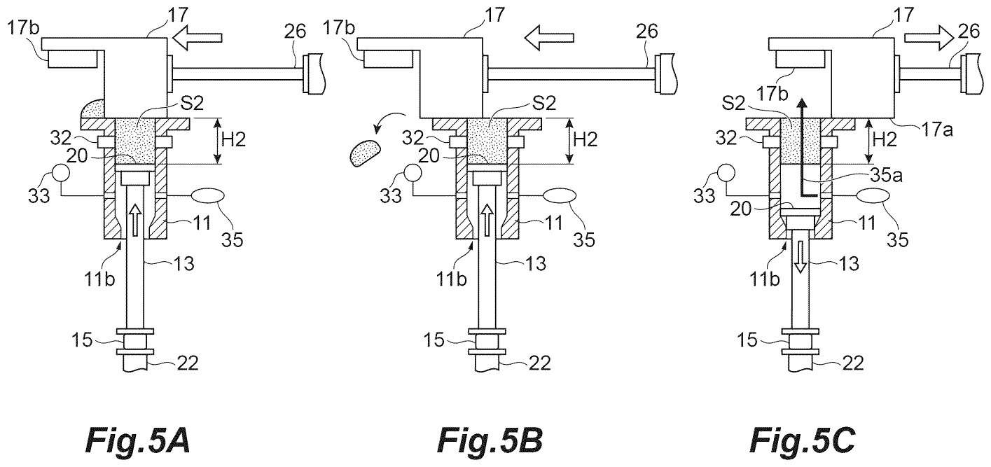

Next, as illustrated in FIG. 5A, the first cylinder control unit 44 raises the piston rod 22 of the first cylinder 14, and applies a prescribed pressure to the foundry sand S1 from the pressurizing plate 20. With this operation, the foundry sand S1 is compressed between the pressurizing plate 20 and the closing surface 17a of the scraper 17, so as to produce a test piece S2 of the foundry sand S1 (step ST6). At this time, the pressure applied from the pressurizing plate 20 to the foundry sand S1 is measured by the load sensor 15, and the measured pressure is returned as a feedback so as to achieve a prescribed pressure.

Next, the CB measurement unit 53 measures a height H2 of the test piece S2 (step ST7). The height H2 of the test piece is obtained based on the position of the pressurizing plate 20 determined by the output of the encoder 23b. The height H2 of the test piece measured in step ST7 is a measured value regarding the height of the test piece S2. Next, the CB measurement unit 53 calculates the CB of the foundry sand S1 (step ST8). The CB of the foundry sand S1 is obtained based on a difference between the height (that is, the initial sand height H1) of the foundry sand S1 before compression and the height H2 of the test piece S2 produced by compressing the foundry sand S1. Specifically, CB [%] is obtained from the following Formula (2).

CB=(H1−H2)/H1×100 (2)

As described above, the CB measurement unit 53 measures the CB of the foundry sand S1 while compressing the foundry sand S1 in the test tube 11 to produce the test piece S2. CB of the foundry sand S1 measured by Formula (2) is a measured value of CB.

Next, the moisture content contained in the test piece S2 is measured by the moisture sensor 32 (step ST9). The moisture content measurement unit 52 acquires information indicating the moisture content contained in the test piece S2 from the moisture sensor 32 and stores the acquired information. The moisture content measurement unit 52 may measure the moisture content before producing the test piece S2.

Next, as illustrated in FIG. 5B, the second cylinder control unit 45 controls the piston rod 26 of the second cylinder 16 to move forward. With this operation, the scraper 17 moves forward to remove the excess foundry sand S1 remaining on the upper surface of the test tube 11.

Next, the air permeability of the test piece S2 is measured (step ST10). As illustrated in FIG. 5C, in order to measure the air permeability of the test piece S2, the first cylinder control unit 44 first lowers the piston rod 22 of the first cylinder 14, and the second cylinder control unit 45 controls the second cylinder 16 to move the scraper 17 to the measurement position. Subsequently, the air permeability measurement unit 54 controls the air source 35 to supply air 35a into the test tube 11 through a gap formed between the pressurizing plate 20 and the test piece S2 so as to pass the air 35a through the test piece S2. The air permeability measurement unit 54 then adjusts the flow rate of the air 35a to achieve a constant pressure in the test tube 11 while monitoring the pressure sensor 33, and obtains the air permeability based on the flow rate of the air 35a when the pressure becomes constant. For example, when the pressure of the internal space 11s measured by the pressure sensor 33 is p and the flow rate of the air 35a is F, the air permeability P is obtained from the following Formula (3).

P=(F×H2)/p (3)

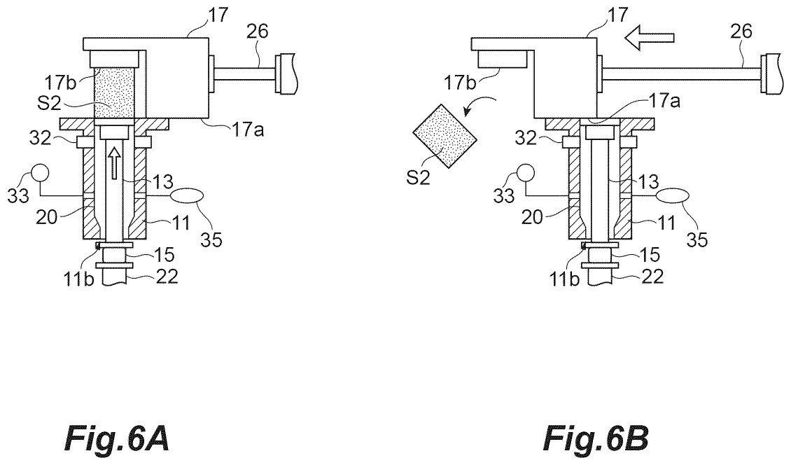

Next, the compressive strength of the test piece S2 is measured (step ST11). As illustrated in FIG. 6A, in order to measure the compressive strength of the test piece S2, the compressive strength measurement unit 55 raises the piston rod 22 of the first cylinder 14 to bring the test piece S2 into contact with the measuring surface 17b. The compressive strength measurement unit 55 further raises the piston rod 22, and applies a prescribed pressure to the test piece S2 from the pressurizing plate 20 to break the test piece S2. Then, the compressive strength measurement unit 55 acquires the load at the breakage of the test piece S2 from the load sensor 15.

Next, the compressive strength measurement unit 55 obtains the compressive strength by dividing the load at the breakage of the test piece S2 by the cross-sectional area of the test piece S2 (that is, the cross-sectional area of an internal space 11c). That is, when the cross-sectional area perpendicular to the axis Z of the internal space 11s of the test tube 11 is A [cm²] and the breaking load of the test piece S2 is W [N], the compressive strength σ [N/cm²] of the test piece S2 is obtained from the following Formula (4).

σ=W/A (4)

After the measurement of the compressive strength σ, as illustrated in FIG. 6B, the second cylinder control unit 45 controls the piston rod 26 of the second cylinder 16 to move forward. With this operation, the test piece S2 is removed.

Next, the compressive strength σ is corrected based on a difference between the target height and the measured value of the height of the test piece S2 (step ST12). Since the initial sand height H1 is obtained from the predictive value of the CB, the height H2 of the test piece S2 produced by compressing the foundry sand S1 can deviate from the target height in some cases. The compressive strength σ depends on the height H2 of the test piece S2. Therefore, when the height H2 of the test piece S2 deviates from the target height, the compressive strength σ obtained by Formula (4) is to be a value deviating from the true compressive strength.

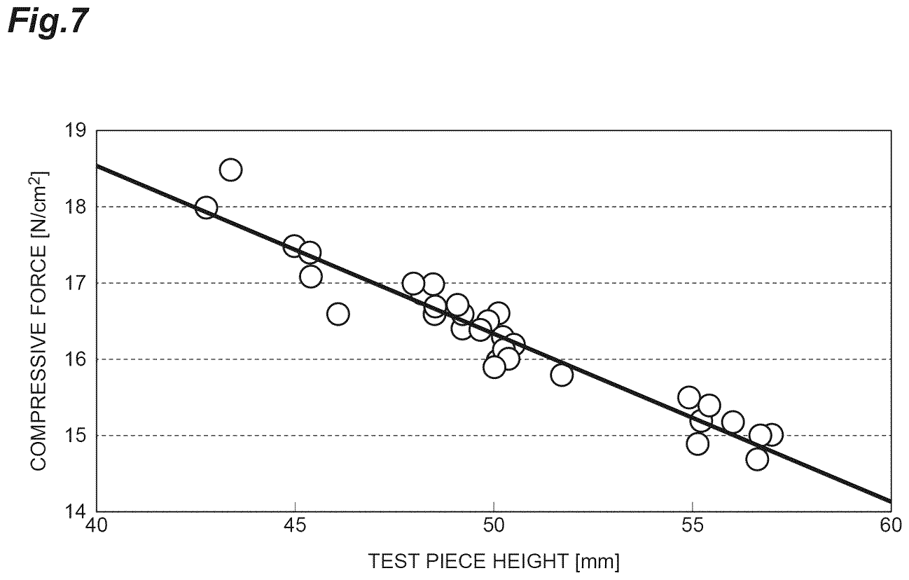

For example, FIG. 7 is a graph illustrating a relationship between the height and the compressive strength of a test piece when the test piece is produced in plurality to have mutually different heights using the same foundry sand and then the compressive strength of each test piece is measured. As illustrated in FIG. 7, the height of the test piece and the compressive strength has a linear relationship. Specifically, the greater the height of the test piece, the smaller the measured value of the compressive strength with respect to the true compressive strength will be; and the smaller the height of the test piece, the greater the measured value of the compressive strength with respect to the true compressive strength will be.

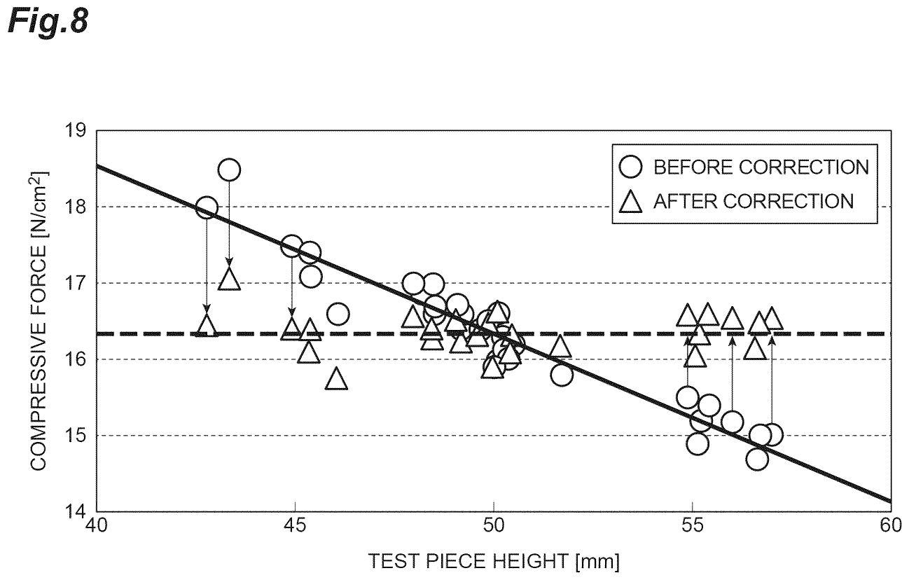

Using this relationship, as illustrated in FIG. 8, the measured value of the compressive strength can be brought close to the true compressive strength by correcting the compressive strength so as to increase the measured value of the compressive strength when the height of the test piece is greater than the target height (50 mm), and correcting the compressive strength so as to decrease the measured value of the compressive strength when the height of the test piece is smaller than the target height (50 mm).

Accordingly, the compressive strength correction unit 56 makes a correction so as to increase the measured compressive strength σ when the height H2 of the test piece S2 measured in step ST7 is greater than the target height, and makes a correction so as to decrease the measured compressive strength σ when the height H2 of the test piece S2 is smaller than the target height. Incidentally, the larger the difference between the height H2 of the test piece S2 and the target height, the larger the correction amount of the compressive strength σ will be. Information measured in the series of steps, such as the CB, the temperature, the moisture content, the air permeability, and the correction value of the compressive strength, is displayed on the display device 60.

As described above, in the inspection method according to the embodiment, the CB of the foundry sand S1 is measured while the test piece S2 is produced by compressing the foundry sand S1 in the test tube 11, making it possible to measure the CB and the compressive strength of the foundry sand S1 using one sample of the foundry sand S1 charged to the test tube 11. This eliminates the need to charge the foundry sand S1 in two sessions, thereby reducing the number of inspection steps. As a result, the inspection time of the foundry sand S1 can be shortened.

In addition, this inspection method corrects the compressive strength so as to increase the measured compressive strength when the height H2 of the test piece S2 is greater than the target height, and corrects the compressive strength so as to decrease the measured compressive strength σ when the height H2 of the test piece S2 is smaller than the target height, making it possible to bring the measured value of compressive strength close to the true compressive strength. As illustrated in Formula (3), the height H2 of the test piece S2 is included in the calculation of the air permeability P. Therefore, even when the height H2 of the test piece S2 deviates from the target height, the influence on the measurement of the air permeability P is small.

Although the inspection method and the inspection device 1 regarding foundry sand according to various embodiments has been described above, the present invention is not limited to the above embodiments, and various modifications can be made without changing the scope and spirit of the invention. That is, it should be noted that the above embodiments are intended for the purpose of illustration and are not intended to limit the scope of the present invention.

For example, although, in the above embodiment, the amount of foundry sand to be charged into the test tube 11 is determined using the predictive value of the CB, the amount of foundry sand to be charged into the test tube 11 may be determined without using the predictive value of the CB. For example, when the predictive value of the amount of foundry sand has been input by the operator, the predictive value of the amount of foundry sand may be determined as the amount of foundry sand to be charged into the test tube 11. In addition, when the CB can be predicted with high accuracy, there is no need to correct the measured value of the compressive strength.

Furthermore, in the above embodiment, the amount of foundry sand to be charged into the test tube 11 is adjusted by controlling the position of the pressurizing plate 20. Alternatively, the amount of foundry sand to be charged into the test tube 11 may be adjusted by adjusting the amount of foundry sand supplied from the chute 12 while maintaining the pressurizing plate 20 at the lowermost position.

The above embodiment performs measurement of the CB, the temperature, the moisture content, the air permeability, and the compressive strength as parameters indicating the characteristics of the foundry sand, but it is only required to measure at least one parameter. For example, in one embodiment, it is also allowable to compress the foundry sand in the test tube 11 to produce a test piece at a target height, and measure only the compressive strength using the test piece. Even when only the compressive strength of the test piece is measured, the height of the test piece can deviate from the target height in some cases. In this case, the measured value of compressive strength can be brought close to the true compressive strength by correcting the measured value of compressive strength.

The present disclosure includes the following contents.

[Clause 1] A foundry sand inspection method according to an aspect includes: charging foundry sand into a test tube; measuring compactability (CB) of the foundry sand while compressing the foundry sand in the test tube to produce a test piece; and compressing the test piece and measuring a compressive strength of the test piece.

In the above-described inspection method, the CB of the foundry sand is measured while the test piece is produced by compressing the foundry sand in the test tube, making it possible to measure the CB and the compressive strength of the foundry sand using one sample of the foundry sand charged to the test tube. This eliminates the need to charge the foundry sand in two sessions, thereby reducing the number of inspection steps. As a result, the inspection time of the foundry sand can be shortened.

[Clause 2] The inspection method according to Clause 1 may further include: acquiring a predictive value of the CB of the foundry sand; and determining, based on the predictive value of the CB, an amount of the foundry sand to be charged into the test tube in order to produce the test piece having a target height. By determining the amount of foundry sand to be supplied into the test tube based on the predictive value of the CB, it is possible to bring the height of the test piece close to the target height. By bringing the height of the test piece close to the target height, it is possible to enhance the measurement accuracy of the compressive strength.

[Clause 3] The inspection method according to Clause 2, further including adjusting a position of a pressurizing plate configured to be movable up and down within the test tube, based on the amount of foundry sand to be charged into the test tube.

[Clause 4] The inspection method according to any one of Clauses 1 to 3 may further include: measuring a height of the test piece; and correcting the compressive strength based on a difference between the target height of the test piece and a measured value of the height of the test piece. The load at the time of compressing and breaking the test piece depends on the height of the test piece. Therefore, occurrence of deviation in an actual height of the test piece from the target height would degrade the measurement accuracy of the compressive strength. In contrast, the compressive strength is corrected, in the present aspect, based on the difference between the target height of the test piece and the measured value of the height of the test piece, the measured value of the compressive strength can be brought close to the true compressive strength.

[Clause 5] The inspection method according to Clause 4, in which, when the measured value of the height of the test piece is greater than the target height, the compressive strength may be corrected to increase the measured compressive strength, and when the measured value of the height of the test piece is smaller than the target height, the compressive strength may be corrected to decrease the measured compressive strength. When the height of the test piece is greater than the target height, the measured value of the compressive strength is lower than the true compressive strength. Therefore, by increasing the compressive strength, the measured value of the compressive strength can be brought close to the true compressive strength. On the contrary, when the height of the test piece is smaller than the target height, the measured value of the compressive strength is higher than the true compressive strength. Therefore, by decreasing the compressive strength, the measured value of the compressive strength can be brought close to the true compressive strength.

[Clause 6] The inspection method according to any one of Clauses 1 to 5 may further include measuring a temperature of foundry sand before producing a test piece.

[Clause 7] The inspection method according to Clause 6 may further include measuring moisture content of the foundry sand after measuring the temperature of the foundry sand.

[Clause 8] The inspection method according to any one of Clauses 1 to 7 may further include measuring air permeability of the test piece before measuring the compressive strength.

[Clause 9] A foundry sand inspection device according to an aspect includes: a test tube extending in an axial direction; a chute configured to supply foundry sand into the test tube; a pressing rod including a pressurizing plate movable in the axial direction in the test tube; a cylinder being connected to the pressing rod and having a piston rod movable forward and backward in the axial direction; a load sensor disposed between the pressing rod and the piston rod; and a control device configured to control operations of the chute and the cylinder, in which the control device includes: a charge control unit configured to control the chute to charge the foundry sand into the test tube; a CB measurement unit configured to measure compactability (CB) of the foundry sand in the test tube while compressing the foundry sand with the pressurizing plate to produce a test piece; and a compressive strength measurement unit configured to measure the compressive strength of the test piece based on a load measured by the load sensor when the test piece is compressed to be broken.

In the above-described inspection device, the CB of the foundry sand is measured while the test piece is produced by compressing the foundry sand in the test tube, making is possible to measure the CB and the compressive strength of the foundry sand using one sample of the foundry sand charged to the test tube. This eliminates the need to charge the foundry sand in two sessions, thereby reducing the number of inspection steps. As a result, the inspection time of the foundry sand can be shortened.

### REFERENCE SIGNS LIST

1 INSPECTION DEVICE
11 TEST TUBE
12 CHUTE
13 PRESSING ROD
15 LOAD SENSOR
18 CONTROL DEVICE
20 PRESSURIZING PLATE
22, 26 PISTON ROD
43 CHARGE CONTROL UNIT
53 CB MEASUREMENT UNIT
55 COMPRESSIVE STRENGTH MEASUREMENT UNIT
S1 FOUNDRY SAND
S2 TEST PIECE

What is claimed is:

1. A foundry sand inspection method comprising:
charging foundry sand into a test tube;
measuring compactability (CB) of the foundry sand while compressing the foundry sand in the test tube to produce a test piece; and
compressing the test piece and measuring a compressive strength of the test piece.

2. The inspection method according to claim 1, further comprising:
acquiring a predictive value of the CB of the foundry sand; and
determining, based on the predictive value of the CB, an amount of the foundry sand to be charged into the test tube to produce the test piece having a target height.

3. The inspection method according to claim 2, further comprising adjusting a position of a pressurizing plate configured to be movable up and down within the test tube, based on the amount of foundry sand to be charged into the test tube.

4. The inspection method according to claim 2, further comprising:
measuring a height of the test piece; and
correcting the compressive strength based on a difference between the target height of the test piece and a measured value of the height of the test piece.

5. The inspection method according to claim 4, wherein, when the measured value of the height of the test piece is greater than the target height, the compressive strength is to be corrected to increase the measured compressive strength, and when the measured value of the height of the test piece is smaller than the target height, the compressive strength is to be corrected to decrease the measured compressive strength.

6. The inspection method according to claim 1, further comprising measuring a temperature of the foundry sand before producing the test piece.

7. The inspection method according to claim 6, further comprising measuring moisture content of the foundry sand after measuring the temperature of the foundry sand.

8. The inspection method according to claim 1, further comprising measuring air permeability of the test piece before measuring the compressive strength.

9. An inspection device comprising:
a test tube extending in an axial direction;
a chute configured to supply foundry sand into the test tube;
a pressing rod including a pressurizing plate movable in the axial direction in the test tube;
a cylinder being connected to the pressing rod and having a piston rod movable forward and backward in the axial direction;
a load sensor disposed between the pressing rod and the piston rod; and
a control device configured to control operations of the chute and the cylinder,
wherein the control device includes:
a charge control unit configured to control the chute to charge the foundry sand into the test tube;
a CB measurement unit configured to measure compactability (CB) of the foundry sand in the test tube while compressing the foundry sand with the pressurizing plate to produce a test piece; and
a compressive strength measurement unit configured to measure the compressive strength of the test piece based on a load measured by the load sensor when the test piece is compressed to be broken.
